(19) United States
(12) Patent Application Publication
(10) Pub. No.: US 2011/0142968 A1
(43) Pub. Date: Jun. 16, 2011

(54) Nutritional supplement

(76) Inventor: David B. Tuchinsky, Hollywood, FL (US)

(21) Appl. No.: 13/030,267

(22) Filed: Feb. 18, 2011

Related U.S. Application Data
(63) Continuation of application No. 12/479,894, filed on Jun. 8, 2009, now abandoned.
(60) Provisional application No. 61/059,415, filed on Jun. 6, 2008.

(51) Int. Cl. A61K 36/84; A61P 29/00

(52) U.S. Cl. 424/733

(57) ABSTRACT

A nutritional supplement useful for promoting good overall health and relieving minor aches and pains associated with inflammation and muscle soreness includes a plurality of active herbal ingredients (turmeric extract, white willow extract, passion flower extract, valerian root extract, and/or licorice root extract), a plurality of non-herbal active ingredients (magnesium glycinate, vitamin B1 (thiamine HCI), vitamin B2 (riboflavin), vitamin B3 (niacinamide), vitamin B6 (pyridoxine HCI), folic acid, vitamin B12, and/or beta carotene). The nutritional supplement may be in the form of an effervescent tablet that is dissolvable in a potable liquid and can include one or more sweeteners and flavorings.

### CROSS-REFERENCE TO RELATED APPLICATIONS

The present application is a continuation of U.S. nonprovisional patent application Ser. No. 12/479,894, filed on Jun. 8, 2009, which claimed priority from U.S. provisional patent application Ser. No. 61/059,415 filed on Jun. 6, 2008. Both applications are herein incorporated in their entirety by reference.

### FIELD OF THE INVENTION

This invention relates to nutritional supplements. More particularly, the invention relates to compositions and methods for supplementing the diet for improving health and ameliorating minor aches and pains due to inflammation or muscle soreness.

### BACKGROUND

A number of over-the-counter drugs exist for treating minor aches and pains. Examples of these include aspirin (acetylsalicylic acid), acetaminophen, ibuprofen, naproxen sodium, and ketoprofen. While usually effective, a large subset of the population is reluctant to take these drugs because of undesired side effects, possible interactions with other medications, or simply because they feel uncomfortable taking unnatural products.

### SUMMARY

The invention is based on the development of a nutritional supplement that includes several different natural ingredients that work together to promote good overall health and also to effectively relieve minor aches and pains due to inflammation or muscle soreness. Compared to conventional analgesic drugs, the nutritional supplement is advantageous because it can be used to relieve minor pain with few or no side effects and is generally acceptable for that segment of the population that is disinclined to use unnatural drugs.

Accordingly, in one aspect, the invention features a nutritional supplement including at least two (e.g., at least 3, 4, or 5) herbal ingredients in an amount effective to reduce minor aches and pains due to inflammation or muscle soreness. The herbal ingredients being selected from the group consisting of: turmeric extract, white willow extract, passion flower extract, valerian root extract, and licorice root extract. The nutritional supplement can be formulated in a single dose including (a) 10-200 mg turmeric extract, 10-150 mg white willow extract, 10-200 mg passion flower extract, 10-200 mg valerian root extract, and 5-75 mg licorice root extract; or (b) about 90 mg turmeric extract, about 50 mg white willow extract, about 80 mg passion flower extract, about 80 mg valerian root extract, and about 25 mg licorice root extract.

The nutritional supplement can further include at least one (e.g., at least 2, 3, 4, or 5) non-herbal ingredient selected from the group consisting of: magnesium glycinate, vitamin B1, vitamin B2, vitamin B3, vitamin B6, folic acid, vitamin B12, and beta carotene. The nutritional supplement can be formulated to include amounts of (a) at least five non-herbal ingredients selected from the group consisting of: 20-300 mg magnesium glycinate, 1-20 mg vitamin B1, 0.5-10 mg vitamin B2, 2-40 mg vitamin B3, 1-20 mg vitamin B6, 10-200 micrograms folic acid, 1-20 mg vitamin B12, and 1-30 mg beta carotene; or about 100 mg magnesium glycinate, about 5 mg vitamin B1, about 2 mg vitamin B2, about 10 vitamin B3, about 5 vitamin B6, about 80 micrograms folic acid, about 5 mg vitamin B12, and about 8 mg beta carotene.

In another aspect the invention features an effervescent tablet including a nutritional supplement including at least two (e.g., at least 3, 4, or 5) herbal ingredients in an amount effective to reduce minor aches and pains due to inflammation or muscle soreness. The herbal ingredients being selected from the group consisting of: turmeric extract, white willow extract, passion flower extract, valerian root extract, and licorice root extract. The effervescent tablet can include about 90 mg turmeric extract, about 50 mg white willow extract, about 80 mg passion flower extract, about 80 mg valerian root extract, and about 25 mg licorice root extract and, optionally, about 100 mg magnesium glycinate, about 5 mg vitamin B1, about 2 mg vitamin B2, about 10 vitamin B3, about 5 vitamin B6, about 80 micrograms folic acid, about 5 mg vitamin B12, and about 8 mg beta carotene. The effervescent tablet can further include at least three (e.g., at least 3, 4, 5, 6, or 7) ingredients selected from the group consisting of malic acid, sodium bicarbonate, potassium bicarbonate, stearic acid, dextrose, stevia, and flavoring.

In another aspect, the invention features a method for reducing minor aches and pains due to inflammation or muscle soreness in a human subject. The method can include the step of administering to the subject a nutritional supplement as set forth above. The method might also include a step of administering to the subject a liquid in which an effervescent tablet of the invention is dissolved.

Unless otherwise defined, all technical terms used herein have the same meaning as commonly understood by one of ordinary skill in the art to which this invention belongs.

The term “about” means plus or minus 50%.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The invention provides compositions and methods for promoting good health and effectively relieving minor aches and pains due to inflammation or muscle soreness. The compositions are typically prepared as effervescent tablets that readily dissolve in water or another potable liquid. Once dissolved, a person seeking analgesia simply drinks the resulting liquid mixture. In alternative embodiments, the active herbal ingredients, and/or active non-herbal ingredients can be used alone or in conjunction with other inactive ingredients/carriers. For example, the active herbal ingredients can be formulated according to the formulas below and included in a capsule (or multiple capsules), powder, or drink with or without the active non-herbal ingredients as formulated below.

The below described preferred embodiments illustrate adaptation of these compositions and methods. Nonetheless, from the description of these embodiments, other aspects of the invention can be made and/or practiced based on the description provided below.

### Compositions

Compositions of the invention include a mixture at least two (e.g., 2, 3, 4, or 5) of the following active herbal ingredients in an amount effective to reduce minor aches and pains due to inflammation or muscle soreness: turmeric extract, white willow extract, passion flower extract, valerian root extract, and licorice root (preferably deglycyrrhizinated) extract. In addition, the foregoing mixture might further include one or more (1, 2, 3, 4, 5, 6, 7, 8, or 9) of the following active non-herbal ingredients: magnesium glycinate, vitamin B1 (thiamine HCI), vitamin B2 (riboflavin), vitamin B3 (niacinamide), vitamin B6 (pyridoxine HCI), folic acid, vitamin B12, and beta carotene. In a preferred embodiment, the compositions of the invention include all or all but 2, 3, 4, or 5 of the foregoing components.

The amount of each ingredient used in a composition can be any sufficient to reduce minor aches and pains due to inflammation or muscle soreness. For a single dose for an average size adult human subject, the amount of the active herbal ingredients might be as follows:

turmeric extract (e.g., 95% curcuminoids)=90 mg

white willow extract (e.g., 25% salicin)=50 mg

passion flower extract=80 mg

valerian root extract (e.g., 0.8% valerenic acid)=80 mg

licorice root (preferably deglycyrrhizinated)=25 mg.

For a single dose for an average size adult human subject, the amount of the active non-herbal ingredient(s) might be as follows:

magnesium glycinate=100 mg

vitamin B1 (thiamine HCI)=5 mg

vitamin B2 (riboflavin)=2 mg

vitamin B3 (niacinamide)=10 mg

vitamin B6 (pyridoxine HCI)=5 mg

folic acid=80 micrograms

vitamin B12 (e.g., 1%)=5 mg

beta carotene=8 mg.

The amounts of active herbal and non-herbal ingredients might vary by plus 10, 20, 30, 40, 50, 60, 70, 80, 90, 100, 150, 200, 300, 400, 500 or more percent by weight; or by minus 10, 20, 30, 40, 50, 60, 70, 80, 90, 91, 92, 93, 94, 95, 96, 97, 98, 99 or more percent by weight.

The compositions of the invention may be administered to a subject in any suitable form. For example, a composition may be in the form of a packaged powder for dissolution in a liquid, an encapsulated powder, a tablet for oral administration, a liquid in which the composition is dissolved (e.g., a pre-packaged sports drink), or an effervescent tablet. The active ingredients described above might be formulated with one or more (e.g., 1, 2, 3, 4, 5, 6, 7 or more non-active ingredients). Examples of non-active ingredients that might be used include an acidulant such as citric acid and/or malic acid (or mixture thereof), sodium bicarbonate (e.g., encapsulated with hydrogenated soybean oil), potassium bicarbonate, stearic acid, a sweetner such as dextrose, stevia or sucralose, and flavoring (e.g., a citrus flavoring such as lemon flavoring). In one embodiment citric acid and/or malic acid makes up between 10-60% (e.g., about 30%) of the composition by weight, sodium bicarbonate makes up between 2.5-25% (e.g., about 10%) of the composition by weight, potassium bicarbonate makes up between 1-10% (e.g., about 3%) of the composition by weight, stearic acid makes up between 1-10% (e.g., about 3%) of the composition by weight, dextrose makes up between 25-85% (e.g., about 65%) of the composition by weight, stevia makes up between 0.05-1% (e.g., about 0.15%) of the composition by weight, and flavoring makes up between 0.001-1% (e.g., about 0.05%) of the composition by weight.

A preferred form of the compositions of the invention is an effervescent tablet that readily dissolves in water (e.g., 1 gram of the composition is completely dissolved in one ounce of room temperature water within 1, 2, 3, or 4 minutes). A sweetened, flavored effervescent tablet may be made by mixing together one or more of the active herbal and non-herbal ingredients described above with non-active ingredients that impart effervescence to the mixture when it is added to an aqueous liquid, and then pressing the mixture into a tablet (e.g., a 1-10 gram tablet such as a 1, 2, 3, 4, 5, 6, 7, 8, 9, or 10 gram tablet).

Non-active ingredients that impart effervescence to the mixture can include an acidulant such as citric acid and/or malic acid (or mixture thereof), sodium bicarbonate (e.g., encapsulated with hydrogenated soybean oil such as at a 15% rate), potassium bicarbonate, stearic acid, dextrose, stevia, and flavoring (e.g., a citrus flavoring such as lemon flavoring). In one example, citric acid and/or malic acid would make up between 10-60% (e.g., between 20-40%, 25-35%, or about 30%) of the composition by weight, sodium bicarbonate would make up between 2.5-25% (e.g., 5-20%, 7.5-12.5%, or about 10%) of the composition by weight, potassium bicarbonate would make up between 1-10% (e.g., 1.5-7%, 2-5%, or about 3%) of the composition by weight, stearic acid would make up between 1-10% (e.g., 1.5-7%, 2-5%, or about 3%) of the composition by weight, dextrose would make up between 25-85% (e.g., 35-80%, 50-75%, 60-70%, or about 65%) of the composition by weight, stevia would make up between 0.05-1% (e.g., 0.08-0.5%, 0.1-0.3%, or about 0.15%) of the composition by weight, and flavoring would make up between 0.001-1% (e.g., 0.005-0.5%, 0.01-0.1%, 0.02-0.07%, or about 0.05%) of the composition by weight.

In an effervescent tablet, turmeric extract (e.g., 95% curcuminoids) could make up between 0.5-5% (e.g., between 1-4%, between 2-3%, between 2.0-2.5%, between 2.1-2.3%, or 2.25%) of the composition by weight, white willow extract (e.g., 25% salicin) could make up between 0.5-4% (e.g., between 1-3%, between 1.5-2.5%, between 1.8-2.2%, between 1.9-2.1%, or 2.0%) of the composition by weight, passion flower extract could make up between 0.5-10% (e.g., between 1-3%, between 1.5-2.5%, between 1.8-2.2%, between 1.9-2.1%, or 2.0%) of the composition by weight, valerian root extract (0.8% valerenic acid) could make up between 0.5-10% (e.g., between 1-3%, between 1.5-2.5%, between 1.8-2.2%, between 1.9-2.1%, or 2.0%) of the composition by weight, and licorice root (preferably deglycyrrhizinated) extract could make up between 0.1-3% (e.g., (e.g., between 0.2-2.0%, between 0.3-1.0%, between 0.4-0.8%, between 0.5-0.7%, or 0.625%) of the composition by weight.

In an effervescent tablet, magnesium glycinate could make up between 0.5-10% (e.g., between 1-5%, between 2-4%, between 2-3%, between 2.3-2.7%, or 2.5%) of the composition by weight, vitamin B1 (thiamine HCI) could make up between 0.01-1% (e.g., between 0.02-0.75%, between 0.05-0.5%, between 0.1-0.3%, between 0.11-0.20%, or 0.125%) of the composition by weight, vitamin B2 (riboflavin) could make up between 0.01-0.25% (e.g., between 0.02-0.2%, between 0.03-0.1%, between 0.04-0.07%, or 0.05%) of the composition by weight, vitamin B3 (niacinamide) could make up between 0.05-2% (e.g., between 0.1-1%, between 0.15-0.5%, between 0.2-0.4%, or 0.25%) of the composition by weight, vitamin B6 (pyridoxine HCI) could make up between 0.01-1% (e.g., between 0.02-0.75%, between 0.05-0.5%, between 0.1-0.3%, between 0.11-0.20%, or 0.125%) of the composition by weight, folic acid could make up between 0.0005-0.02% (e.g., between 0.001-0.01%, between 0.0015-0.005%, between 0.002-0.004%, or 0.0025%) of the composition by weight, vitamin B12 (1%) could make up between 0.01-1% (e.g., between 0.02-0.75%, between 0.05-0.5%, between 0.1-0.3%, between 0.11-0.20%, or 0.125%) of the composition by weight, and beta carotene (1%) could make up between 0.05-1.5% (e.g., between 0.1-1.0%, between 0.125-0.5%, between 0.15-0.25%, or 0.20%) of the composition by weight.

### Methods of Use

The invention features a method for reducing minor aches and pains due to inflammation or muscle soreness (e.g., back pain). This method includes the step of administering to a subject having minor aches and pains due to inflammation or muscle soreness a sufficient dosage of a composition of the invention to alleviate the aches and pain. The compositions of the invention are preferably administered orally. For example, an effervescent tablet of the invention can be dissolved in a potable liquid such as water and the resulting liquid can be drunk by a subject.

The dosage of the compositions of the invention will vary according to the concentration of active ingredients in a given composition. Generally, a dosage of about 1-10 grams (e.g., 0.5, 1, 2, 3, 4, 5, 6, 7, 8, 9, 10, or 11) per day is expected to be useful for alleviating in human subjects minor pain from inflammation or muscle soreness. The compositions might be administered as needed for pain relief or on a regular basis for chronic pain (e.g., once a day, twice a day, every two days, or every three days). The active ingredients are preferably included at a concentration effective to reduce a subject's pain by at least 10% (e.g., at least 20, 30, 40, or 50%).

### EXAMPLES

### Example 1

### Effervescent Tablet

The following ingredients were added to a Patterson double cone mixer and blended for 20 minutes:

| | | |
|---|---|---|
| Magnesium Glycinate (15%) | 2.46 | lb |
| Turmeric Extract (95% Curcuminoids) | 2.22 | lb |
| White Willow extract (25% Salicin) | 1.97 | lb |
| Passion Flower extract | 1.97 | lb |
| Valerian Root extract (0.8% SE for Valerenic Acid) | 1.97 | lb |
| Licorice Root extract/deglycyrrhizinated | 0.617 | lb |
| Vitamin B1 (Thiamine HCl) | 0.12 | lb |
| Vitamin B2 (Riboflavin) | 0.05 | lb |
| Vitamin B3 (Niacinamide) | 0.246 | lb |
| Vitamin B6 (Pyridoxine HCl) | 0.12 | lb |
| Folic Acid | 0.002 | lb |
| Vitamin B12 (1%) | 0.123 | lb |
| Beta Carotene (1%) | 0.2 | lb |
| Citric Acid | 25 | lb |
| Sodium bicarbonate | 8 | lb |
| Potassium Bicarbonate | 2 | lb |
| Stearic Acid | 2 | lb |
| Dextrose (Can Tab from Penwalt) | 51.532 | lb |
| Stevia (90% Steviocides) | 0.1 | lb |
| Lemon Flavor (from Metaugus) | 0.04 | lb |

The resulting powder was added to a Stokes D3 tablet press and 4 gram disc-shaped tablets were made using a 3.3 on tolerance pressure. The tablets were then de-dusted and packaged. Tablets exhibited a dissolution rate of 3 minutes or less.

### Example 2

### Effervescent Nutritional Drink

Tablets were prepared as in Example 1. A 4 gram tablet was added to about eight ounces of water at room temperature. The tablet was completely dissolved within 4 minutes, leaving an effervescent yellow-green colored essentially transparent drink with a citrus flavor.

### Example 3

### Back Pain

One 4 gram tablet as in Example 1 (except that sucralose was used in place of stevia) was added to about six to eight ounces of water at room temperature. Twenty subjects were instructed to drink the resulting liquid 30-60 minutes before bedtime or during the day (e.g., at lunch time). Most subjects indicated they only took the drink one time during a 24 hour period and most subjects also indicated that they took the drink at bedtime. The results of a survey of the subjects are in Table 1 presented below.

TABLE 1

| | NO. OF | NO. OF | ABSTEN- |
|---|---|---|---|
| SURVEY QUESTIONS | “YES” | “NO” | TIONS |
| Do you now or did you | 20 | 0 | 0 |
| have back pain? | | | |
| Do you now or did you | 16 | 4 | 0 |
| have muscle spasm? | | | |
| Do you suffer from | 14 | 6 | 0 |
| stress and tension? | | | |
| Did you take the | 20 | 0 | 0 |
| nutritional | | | |
| supplement? | | | |
| Did it help? | 20 | 0 | 0 |
| Were the results of | 20 | 0 | 0 |
| the nutritional | | | |
| supplement quick? | | | |
| If you took the | 3 | 0 | 17 |
| nutritional | | | |
| supplement more than | | | |
| once did you notice a | | | |
| compounding effect of | | | |
| relief? | | | |
| Would you recommend | 20 | 0 | 0 |
| the nutritional | | | |
| supplement to others? | | | |
| Do you feel this | 20 | 0 | 0 |
| product should be | | | |
| made available to the | | | |
| general public? | | | |

### Example 4

### Effervescent Tablet

The following ingredients were added to a Patterson double cone mixer and blended for 20 minutes:

| | | |
|---|---|---|
| Magnesium Glycinate (15%) | 2.46 | lb |
| Turmeric Extract (95% Curcuminoids) | 2.22 | lb |
| White Willow extract (25% Salicin) | 1.23 | lb |
| Passion Flower extract | 1.97 | lb |
| Valerian Root extract (0.8% SE for Valerenic Acid) | 1.97 | lb |
| Licorice Root extract/deglycyrrhizinated | 0.617 | lb |
| Vitamin B1 (Thiamine HCl) | 0.12 | lb |
| Vitamin B2 (Riboflavin) | 0.05 | lb |
| Vitamin B3 (Niacinamide) | 0.246 | lb |
| Vitamin B6 (Pyridoxine HCl) | 0.12 | lb |
| Folic Acid | 0.002 | lb |
| Vitamin B12 (1%) | 0.123 | lb |
| Beta Carotene (1%) | 0.2 | lb |
| Malic Acid | 25 | lb |
| Encapsulated sodium bicarbonate | 8 | lb |
| (encapsulated 15% hydrogenated soybean oil) | | |
| Potassium Bicarbonate | 2 | lb |
| Stearic Acid | 2 | lb |
| Dextrose (Can Tab from Penwalt) | 51.532 | lb |
| Stevia (90% Steviocides) | 0.1 | lb |
| Lemon Flavor (from Metaugus) | 0.04 | lb |

The resulting powder was added to a Stokes D3 tablet press, specific to the particular tablet shape and size (a 4 gram disc shaped round die) to produce a 4 gram tablet (single dose size). The machine was adjusted to a 3.3 on tolerance pressure. The resulting tablet has a dissolution rate in water of about 45 seconds. Tablets are de-dusted in the standard manner and packaged (e.g., vacuum sealed).

### Example 5

### Nutritional Supplement Tablet

A single dosage of a nutritional supplement contains:

| | | |
|---|---|---|
| Magnesium (from Magnesium Glycinate) | 15 | mg |
| Turmeric Extract (95% Curcuminoids) | 120 | mg |
| White Willow extract (25% Salicin) | 80 | mg |
| Passion Flower extract (3.5% flavanoids) | 40 | mg |
| Valerian Root extract (0.8% Valerenic Acid) | 80 | mg |
| Licorice Glycyrrhizinic acid NLT 3% | 25 | mg |
| Vitamin B1 (as Thiamine mononitrate 98% DC G) | 2 | mg |
| Vitamin B2 (Riboflavin 5′-phosphate) | 1 | mg |
| Vitamin B3 (Niacinamide) | 10 | mg |
| Vitamin B6 (Pyridoxine HCl 98% DC) | 2 | mg |
| Vitamin B12 (1%) | 0.025 | mg |
| Folic Acid (10% trituration) | 0.1 | mg |
| Citric Acid (anhydrous fine granular) | 2112.5 | mg |
| Sodium bicarbonate (fine granular) | 1625 | mg |
| Dextrose DC | 3162 | mg |
| Flavor Orange Nat. #421 | 381.9 | mg |
| Stearic acid powder NF | 75 | mg |
| Sucralose | 10.5 | mg |
| N & A Lemon Flavor | 32.5 | mg |
| Prosweet N&A conc. (flavor) MM52 | 130 | mg |
| (Virginia Dare, USA). | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nutritional supplement comprising at least two herbal ingredients in an amount effective to reduce minor aches and pains due to inflammation or muscle soreness, the herbal ingredients being selected from the group consisting of: turmeric extract, white willow extract, passion flower extract, valerian root extract, and licorice root extract.

2. The nutritional supplement of claim 1, comprising at least three herbal ingredients in an amount effective to reduce minor aches and pains due to inflammation or muscle soreness, the herbal ingredients being selected from the group consisting of: turmeric extract, white willow extract, passion flower extract, valerian root extract, and licorice root extract.

3. The nutritional supplement of claim 1, comprising at least four herbal ingredients in an amount effective to reduce minor aches and pains due to inflammation or muscle soreness, the herbal ingredients being selected from the group consisting of: turmeric extract, white willow extract, passion flower extract, valerian root extract, and licorice root extract.

4. The nutritional supplement of claim 1, comprising turmeric extract, white willow extract, passion flower extract, valerian root extract, and licorice root extract.

5. The nutritional supplement of claim 4, wherein the nutritional supplement is formulated in a single dose comprising 10-200 mg turmeric extract, 10-150 mg white willow extract, 10-200 mg passion flower extract, 10-200 mg valerian root extract, and 5-75 mg licorice root extract.

6. The nutritional supplement of claim 4, wherein the nutritional supplement is formulated in a single dose comprising about 90 mg turmeric extract, about 50 mg white willow extract, about 80 mg passion flower extract, about 80 mg valerian root extract, and about 25 mg licorice root extract.

7. The nutritional supplement of claim 1, further comprising at least one non-herbal ingredient selected from the group consisting of: magnesium glycinate, vitamin B1, vitamin B2, vitamin B3, vitamin B6, folic acid, vitamin B12, and beta carotene.

8. The nutritional supplement of claim 2, further comprising at least two non-herbal ingredients selected from the group consisting of: magnesium glycinate, vitamin B1, vitamin B2, vitamin B3, vitamin B6, folic acid, vitamin B12, and beta carotene.

9. The nutritional supplement of claim 3, further comprising at least three non-herbal ingredients selected from the group consisting of: magnesium glycinate, vitamin B1, vitamin B2, vitamin B3, vitamin B6, folic acid, vitamin B12, and beta carotene.

10. The nutritional supplement of claim 4, further comprising at least four non-herbal ingredients selected from the group consisting of: magnesium glycinate, vitamin B1, vitamin B2, vitamin B3, vitamin B6, folic acid, vitamin B12, and beta carotene.

11. The nutritional supplement of claim 5, further comprising amounts of at least five non-herbal ingredients selected from the group consisting of: 20-300 mg magnesium glycinate, 1-20 mg vitamin B1, 0.5-10 mg vitamin B2, 2-40 mg vitamin B3, 1-20 mg vitamin B6, 10-200 micrograms folic acid, 1-20 mg vitamin B12, and 1-30 mg beta carotene.

12. The nutritional supplement of claim 6, further comprising about 100 mg magnesium glycinate, about 5 mg vitamin B1, about 2 mg vitamin B2, about 10 vitamin B3, about 5 vitamin B6, about 80 micrograms folic acid, about 5 mg vitamin B12, and about 8 mg beta carotene.

13. An effervescent tablet comprising the composition of claim 1.

14. The effervescent tablet of claim 13, further comprising the composition of claim 6.

15. The effervescent tablet of claim 13, further comprising the composition of claim 12.

16. The effervescent tablet of claim 13, further comprising at least three ingredients selected from the group consisting of malic acid, sodium bicarbonate, potassium bicarbonate, stearic acid, dextrose, stevia, and flavoring.

17. The effervescent tablet of claim 14, further comprising at least five of the ingredients selected from the group consisting of malic acid, sodium bicarbonate, potassium bicarbonate, stearic acid, dextrose, stevia, and flavoring.

18. The effervescent tablet of claim 15, further comprising malic acid, sodium bicarbonate, potassium bicarbonate, stearic acid, dextrose, stevia, and flavoring.

19. A method for reducing minor aches and pains due to inflammation or muscle soreness in a human subject, the method comprising the step of administering to the subject the nutritional supplement of claim 1.

20. The method of claim 19, the method comprising the step of administering to the subject a liquid in which the effervescent tablet of claim 18 is dissolved.
